(19) United States
(12) Patent Application Publication
(10) Pub. No.: US 2026/0308191 A1
(43) Pub. Date: Oct. 8, 2026

(54) STOICHIOMETRIC ORGANONICKEL COMPLEXES FOR RAPID MOLECULAR DIVERSIFICATION

(71) Applicant: Ohio State Innovation Foundation, Columbus, OH (US)

(72) Inventors: Christo SEVOV, Columbus, OH (US); Taylor HAMBY, Chapel Hill, NC (US); Long DINH, Columbus, OH (US); Hunter STARBUCK, Columbus, OH (US)

(21) Appl. No.: 19/164,458

(22) PCT Filed: Mar. 14, 2024
(86) PCT No.: PCT/US24/19847

§ 371 Date: Sep. 11, 2025

Related U.S. Application Data
(60) Provisional application No. 63/490,039, filed on Mar. 14, 2023.

(51) Int. Cl. C07F 15/04

(52) U.S. Cl. CPC C07F 15/045

(57) ABSTRACT

Disclosed herein are kits for rapidly building molecular libraries. The kits include at least two separate containers, each including an organonickel compound.

### CROSS-REFERENCE TO RELATED APPLICATION

This application claims the benefit of U.S. Provisional Application 63/490,039, filed Mar. 14, 2023, the contents of which are hereby incorporated in its entirety.

### STATEMENT OF GOVERNMENT SUPPORT

This invention was made with government support under R35GM138373 awarded by the National Institutes of Health. The government has certain rights in the invention.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions, Wiley Interscience, New York, 1981; Wilen et al., Tetrahedron 33:2725 (1977); Eliel, E. L. Stereochemistry of Carbon Compounds, McGraw-Hill, NY, 1962; and Wilen, S. H., Tables of Resolving Agents and Optical Resolutions p. 268, E. L. Eliel, Ed., Univ. of Notre Dame Press, Notre Dame, IN 1972. The invention additionally encompasses compounds as individual isomers substantially free of other isomers, and alternatively, as mixtures of various isomers.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₅, and C₅₋₆ alkyl.

The term “alkyl” refers to a radical of a straight-chain hydrocarbon, branched hydrocarbon, or carbocyclic group. In some embodiments, an alkyl group has 1 to 30 carbon atoms (“C₁₋₃₀ alkyl”). In some embodiments, an alkyl group has 1 to 25 carbon atoms (“C₁₋₂₅ alkyl”). In some embodiments, an alkyl group has 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 10 carbon atoms (“C₁₋₁₀ alkyl”). An alkyl group can be saturated or unsaturated, i.e., an alkenyl, alkynyl, cycloalkenyl, or cycloalkynyl group. Unless specified to the contrary, an “alkyl” group includes both saturated alkyl groups and unsaturated alkyl groups.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group has 1 to 20 carbon atoms and 1 or more heteroatoms (“heteroC₁₋₂₀ alkyl”). In some embodiments, a heteroalkyl group has 1 to 18 carbon atoms and 1 or more heteroatoms (“heteroC₁₋₁₈ alkyl”). In some embodiments, a heteroalkyl group has 1 to 16 carbon atoms and 1 or more heteroatoms (“heteroC₁₋₁₆ alkyl”). In some embodiments, a heteroalkyl group has 1 to 14 carbon atoms and 1 or more heteroatoms (“heteroC₁₋₁₄ alkyl”). In some embodiments, a heteroalkyl group has 1 to 12 carbon atoms and 1 or more heteroatoms (“heteroC₁₋₁₂ alkyl”). In some embodiments, a heteroalkyl group has 1 to 10 carbon atoms and 1 or more heteroatoms (“heteroC₁₋₁₀ alkyl”). In some embodiments, a heteroalkyl group has 1 to 8 carbon atoms and 1 or more heteroatoms (“heteroC₁₋₈ alkyl”). In some embodiments, a heteroalkyl group has 1 to 6 carbon atoms and 1 or more heteroatoms (“heteroC₁₋₆ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

The term “halo” or “halogen” refers to fluorine (fluoro, —F), chlorine (chloro, —C₁), bromine (bromo, —Br), or iodine (iodo, —I).

As used herein, a chemical bond depicted: [symbol] represents either a single, double, or triple bond, valency permitting. By way of example,

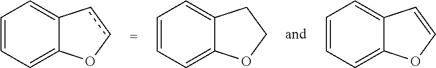

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Unless stated to the contrary, a substituent drawn without explicitly specifying the point of attachment indicates that the substituent may be attached at any possible atom. For example, in a benzofuran depicted as:

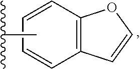

the substituent may be present at any one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

Compounds disclosed herein may be provided in the form of salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

Disclosed herein are kits containing multiple organonickel compounds. The organonickel compounds are substrates for cross-coupling reactions with electrophiles under mild conditions, with wide substrate tolerability. As such, the kits are useful in drug discovery contexts for the rapid creation of compound libraries. In some implementations the kit will include at least two substrate containers,
wherein
the first substrate container includes an organonickel compound of Formula (1):

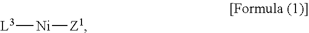

the second substrate container includes an organonickel compound of Formula (2):

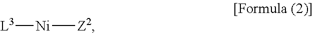

Z¹ has the formula:

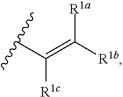

Z² has the formula:

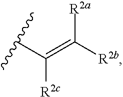

wherein R^{1a}, R^{1b}, R^{1c}, R^{2a}, R^{2b}, and R^{2c} are independently selected from hydrogen, halo, alkyl, heteroalkyl, aryl, or heteroaryl;
wherein Z¹ and Z² are not the same; and
L³-Ni has the formula:

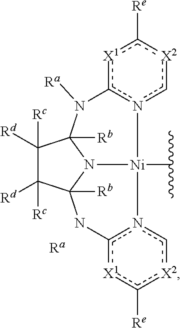

wherein:
a) R^{d} and R^{e} together form a fused benzo ring, and R^{a} and R^{b} together form a double bond; or
b) R^{d} is hydrogen, R^{b} and R^{e} together form a double bond, and R^{a} is C₁₋₄alkyl;
   X¹ is N, O, or CR^{f};
   X² is null, N, or CR^{g}; and
   R^{e} is H, C₁₋₈alkyl, C₁₋₈hydroxylakyl, OC₁₋₈alkyl OC₁₋₈hydroxyalkyl, halo, O(CH₂CH₂O)ₙR^{peg} or N(C₁₋₈alkyl) 2; wherein n is 1-20, 2-10, 2-5, 5-10, or 10-20, and R^{peg} is H or CH₃;
   R^{f} is H, C₁₋₈alkyl, OC₁₋₈alkyl, halo, or N(C₁₋₈alkyl)₂; and
   R^{g} is H, C₁₋₈alkyl, OC₁₋₈alkyl, halo, or N(C₁₋₈alkyl)₂;
   wherein any two or more of R^{e}, R^{f}, and R^{g} may together form a bond.

The skilled person understands that for embodiments in which R^{d} and R^{c} together form a fused benzo ring, the complex has the structure (optional benzo substituents not shown):

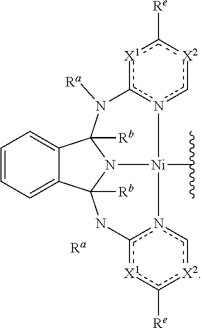

It is also understood that when for embodiments in where R^{a} and R^{b} together form a double bond, or R^{b} and R^{e} together form a double bond, the relevant R groups are those that are adjacent to one another.

The kit can include more than two organonickel compounds, each in a separate substrate container. In some implementations, the kit can include a third substrate container that includes an organonickel compound of Formula (3):

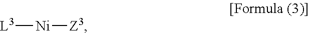

wherein Z³ has the formula:

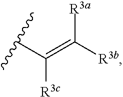

wherein R^{3a}, R^{3b}, and R^{3c} are independently selected from hydrogen, halo, alkyl, heteroalkyl, aryl, or heteroaryl;
wherein each of Z¹, Z², and Z³ are different.

In some implementations, the kit can include a fourth substrate container that includes an organonickel compound of Formula (4):

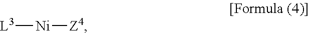

wherein Z⁴ has the formula:

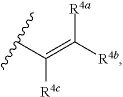

wherein R^{4a}, R^{4b}, and R^{4c} are independently selected from hydrogen, halo, alkyl, heteroalkyl, aryl, or heteroaryl;
wherein each of Z¹, Z², Z³, and Z⁴ are different.

In some implementations the kit can include a fifth substrate container that includes an organonickel compound of Formula (5):

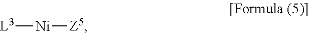

wherein Z⁵ has the formula:

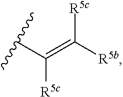

wherein R^{5a}, R^{5b}, and R^{5c} are independently selected from hydrogen, halo, alkyl, heteroalkyl, aryl, or heteroaryl;
wherein each of Z¹, Z², Z³, Z⁴, and Z⁵ are different.

There is no particular limitation as to the number of different substrate containers present in the kit. In some implementations, the kit includes at least 10 different substrate containers, at least 25 different substrate containers, at least 50 different substrate containers, or at least 100 different substrate containers. In certain implementations, each substrate container includes a unique (relative to the kit) organonickel compound, i.e., each organonickel compound is different.

The substrate containers are typically inert containers, for example a reaction vessel to which other reagents can be added. The organonickel compounds can be provided in substantially isolated form, e.g., at least 90%, 95%, 98%, 99%, or 99.5% of the solids contents of the container are the organonickel compound. The organonickel compounds can be provided under an inert atmosphere, e.g., N₂ or Ar, or may be provided under air. In certain implementations, the organonickel compounds can be provided in a solution with organic solvent, optionally in combination with one or more additional components as described herein.

A variety of tridentate ligands (L³) may be used with the organonickel compounds. In some implementations, X¹ is O and X² is null, i.e., oxazole rings at the peripheral rings. In other implementations, X¹ is N and X² is CR^{g}, i.e., pyrimidine rings at the peripheral rings. In certain preferred implementations, X¹ is CR^{f} and X² is CR^{g}, i.e., pyridine rings at the peripheral rings. In some implementations, L³Ni has the formula:

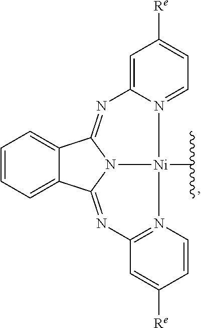

wherein R^{e} is H or C₁-4alkyl;
The organonickel compounds can include a wide variety of substrates (e.g., Z¹, Z², Z³, Z⁴, Z⁵ groups, etc.). The organonickel compounds (and cross-coupling reactions) are tolerant of a wide variety of functional groups, including carboxylic acids and esters, alcohols, amines, halogens and the like. There is no particular limitation as to the molecular size or complexity of the R^{xa}, R^{xb}, and R^{xc} groups (as used herein, R^{xa} collectively refers to all of the R^{1a}, R^{2a}, R^{3a}, etc groups). For example, one or more of these groups can be based on complex chemotherapeutics and antibiotics, e.g, vinca alkaloids, taxanes, sirolimus, vancomycins, camptothecins, eribulins, doxorubicins, lipopeptide antibiotics, and the like.

Exemplary Z groups include:

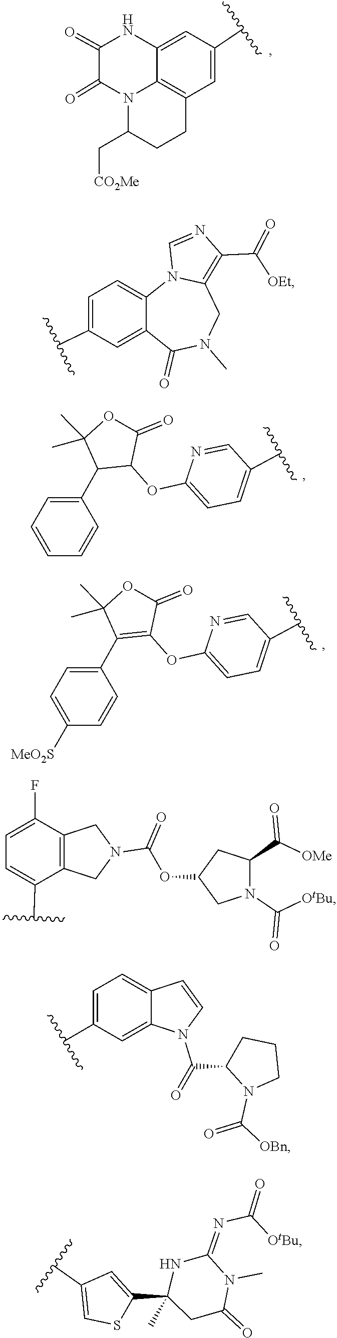

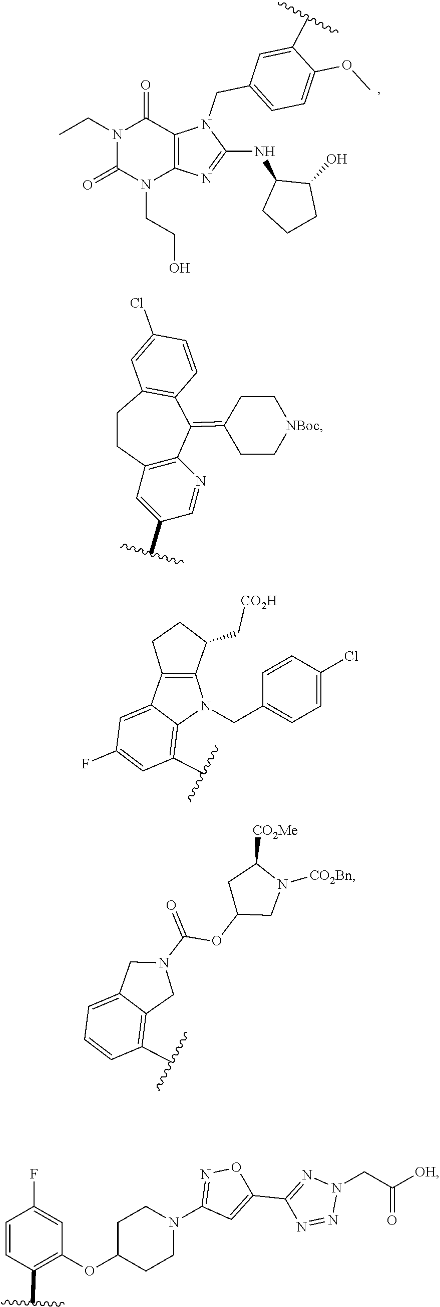

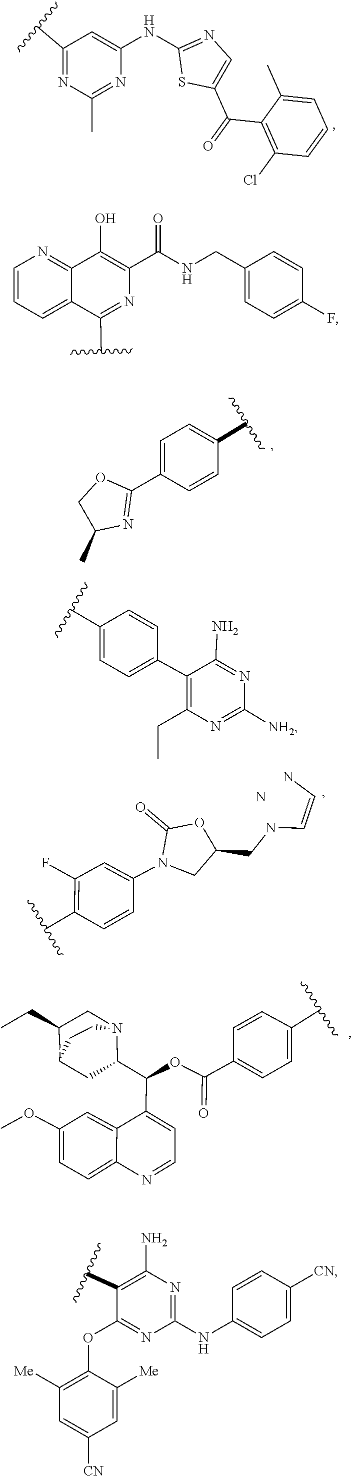

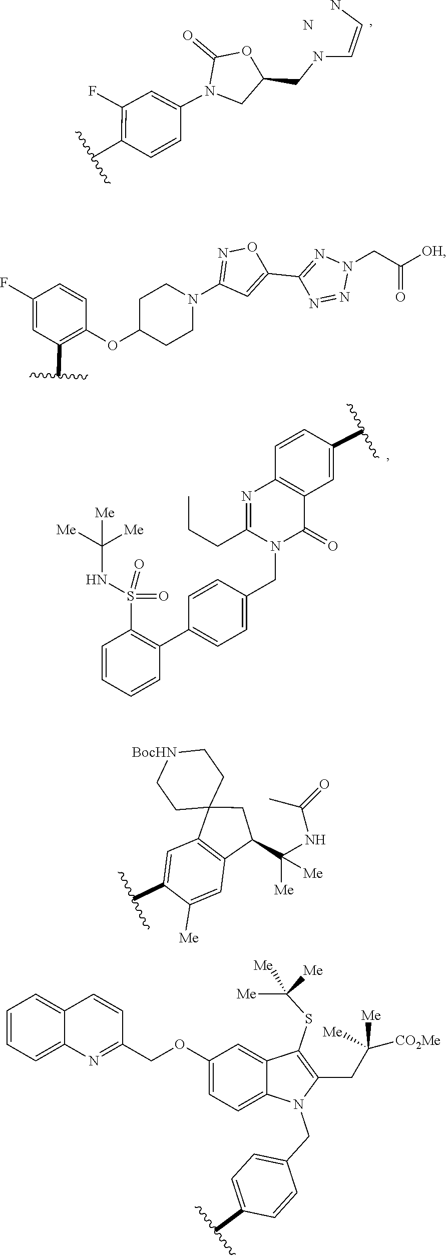

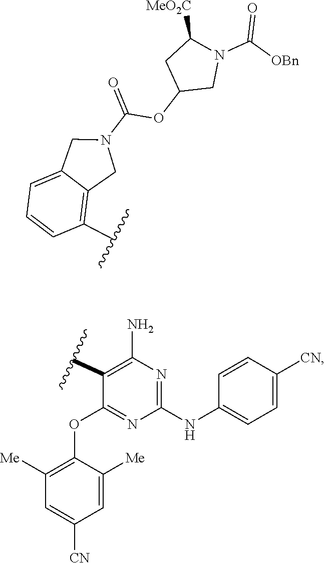

The kits can include at least 5 different compounds, at least 10 different compounds, at least 20 different compounds, at least 50 different compounds, or at least 100 different compounds, each provided separately in an inert container.

In certain implementations the kits can include one or more of the following complexes:

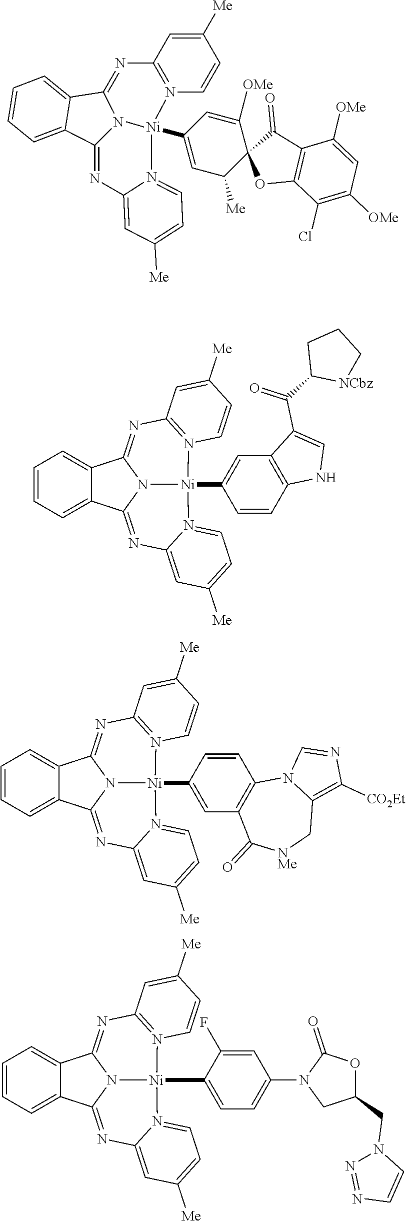

In certain implementations, each compound in the kit will include the same L³-Ni system. However, in other embodiments, the L³ group may vary from compound to compound in view of the specific Z group in each compound.

In some implementations, the kits also include one or more electron donors in one or more electron container. In certain implementations the electron donor is a Ni salt, for example a reduced Ni⁺¹ salt. In some embodiments, the electron donor may be provided in an amount to supply at least one equivalent of electrons to the organonickel compound. In some implementations, the electron donor is provided in excess relative the organonickel compound.

In some implementations the electron donor is a nickel complex having the formula of Formula (e):

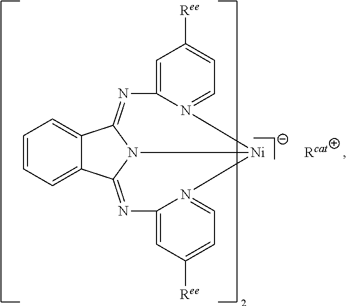

wherein R^{cat} represents an ion in the +1 oxidation state, and R^{ee} is H, C₁₋₄alkyl, OC₁₋₄alkyl, halo, or (CH₂CH₂O)ₙR^{peg}— or N(C₁₋₈alkyl)₂; wherein n is 1-20, 2-10, 2-5, 5-10, or 10-20, and R^{peg} is H or CH₃;

In certain implementations, R^{ee} is methyl, and R^{cat} is selected from Li, Na, or K.

In certain implementations, the kit may include the electron donor in a single electron container, wherein the container includes sufficient compound to conduct coupling reactions with each of the compounds provided in the kit. In other implementations, each organonickel compound is paired with a separate electron container containing sufficient electron donor for the amount of organonickel compound in the pair. In some implementations, each container of an organonickel compound will be matched with an electron container that includes the electron donor. In certain implementations, the electron donor will be present in the electron container in an amount that is 1-10 eq. relative to the amount of paired organonickel compound. In some implementations, the electron donor will be present in an amount that is from 1-3 eq., 2-5 eq., 4-7 eq., or 5-10 eq.

In certain implementations the kits may further include a compound of Formula (L):

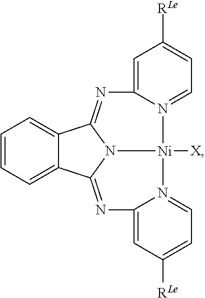

wherein X is halo or OAc; and R^{Le} is H, C₁₋₄alkyl, OC₁₋₄alkyl, halo, or N(C₁₋₄alkyl)₂. In some implementations, R^{Le} is methyl.

In certain implementations, the kit may include the compound of Formula (L) in a single container, wherein the container includes sufficient compound to conduct coupling reactions with each of the organonickel compounds provided in the kit. In other implementations, each substrate container is paired with a separate ligand container including the compound of Formula (L) in an amount sufficient for an individual coupling reaction (based on the amount of organonickel compound present in the substrate container). In some implementations, each substrate container will be matched with a separate ligand container holding the compound of Formula (L). In other implementations, the compound of Formula (L) and the organonickel compound may be provided in the same container. In some implementations, the compound of Formula (L) will be present in an amount that is no more than 0.1 eq. of the amount of organonickel compound (i.e., the equivalents range from 0<compound of Formula (L)≤0.1), no more than 0.05 eq. of the amount of organonickel compound (i.e., the equivalents range from 0<compound of Formula (L)≤0.05), no more than 0.01 eq. of the amount of organonickel compound (i.e., the equivalents range from 0<compound of Formula (L)≤0.01), no more than 0.005 eq. of the amount of organonickel compound (i.e., the equivalents range from 0<compound of Formula (L)≤0.005), or no more than 0.001 eq. of the amount of organonickel compound (i.e., the equivalents range from 0<compound of Formula (L)≤0.001).

The kits may be advantageously deployed to rapidly build chemical libraries. In some implementations, the compounds of Formula (1) may be coupled with a variety of different electrophiles having the formula R—X³, RO—X³, or R₂N—X³ wherein R is a sp³ hybridized carbon and X³ is a leaving group. Exemplary X³ groups include halides, especially bromine and iodine, pyridinium complexes, hydroxysuccinates, ethers, triflates, mesylates, tosylates, and the like.

In some implementations, the electron donor can be added to a reaction mixture including the organonickel compound, the electrophile, optionally in the presence of the compound of Formula (L), in a suitable solvent. After the reaction is complete, the coupled product can be isolated using conventional techniques.

### EXAMPLES

The following examples are for the purpose of illustration of the invention only and are not intended to limit the scope of the present invention in any manner whatsoever.

### Example 1: Electrochemical Synthesis of Organonickel Complexes

In a nitrogen-filled glove-box, a 12 mL screw-cap vial was charged with LiBr (108 mg, 1.25 mmol, 5 equiv.), (MeBPI)NiOAc (111 mg, 0.250 mmol, 1 equiv.), aryl bromide (0.375 mmol, 1.5 equiv.), DMF (5 mL), and a magnetic stir bar. This yellow slurry was allowed to stir at room temperature for 10 minutes. A Ni foam cathode and a Zn anode were inserted into the test tube, the leads were pierced through a septum cap, and the vial was sealed with a screw-cap. The sealed test tube was removed from the glovebox. The reaction mixture was stirred while a reductive, constant current was applied at the Ni cathode (3 mA, 13.4 mAh, 2.0 equiv e). After electrolysis, the reaction mixture was transferred to a 40 mL scintillation vial equipped with a stir bar. To this stirring mixture was slowly added degassed water (~30 mL) until a reddish-orange solid precipitated. The resulting slurry was allowed to stir for 10 minutes at room temperature and then 10 minutes at 0° C. for 10 minutes. The precipitated solid was collected by vacuum filtration and washed with water (100 mL) and ice-cold Et₂O (2×5 mL). The resulting orange solid was dissolved in DCM (20 mL) to afford a deep red solution and washed with water (2×30 mL) and brine (30 mL). The red organic layer was dried over Na₂SO₄, filtered, and concentrated by rotary evaporation. The resulting red solid was dissolved in Et₂O. Slow addition of pentane to this stirring mixture affords the product as a dark red or orange solid. Recrystallization can be performed to enhance purity.

### Alternative Procedure

In a nitrogen-filled glove-box, a 12 mL screw-cap vial was charged with (BPI) Ni—OAc (67 mg, 0.15 mmol, 1 equiv.), C (sp²) electrophile (0.225 mmol, 1.5 equiv.), LiBr (65 mg, 0.75 mmol, 5 equiv.), DMF (3 mL), and a magnetic stir bar. This yellow slurry was allowed to stir at room temperature for 10 minutes. A Ni foam cathode and a Zn anode were inserted into the test tube. The leads were pierced through a septum cap, and the vial was sealed with a screw-cap. The sealed test tube was removed from the glovebox. The reaction mixture was stirred while a reductive, constant current was applied at the Ni cathode (3 mA, 8.04 mAh, 2.0 equiv. e″).

To the resulting deep red solution was added the electrophilic coupling partner as a DMF solution (0.750 mmol, 5 equiv, 1.5 M). The reaction mixture was stirred while a reductive, constant current was applied at the Ni cathode (3 mA, 8.04 mAh, 2 equiv. e) After electrolysis, the product was extracted from the crude reaction mixture with Et₂O (3×25 mL) and water (50 mL). The organic layers were combined and washed with brine (50 mL). The organic layer was dried over Na₂SO₄, filtered, and concentrated by rotary evaporation. The product was purified by flash chromatography on silica gel. Variations to this general procedure and specific conditions for isolation are detailed for each substrate below.

### Example 2: Stoichiometric Reductive Coupling

Stock solutions of each individual reagent were prepared in a nitrogen-filled glove-box. A 20 mL screw-cap vial was charged with Ni-Aryl (130. mg, 0.254 mmol), dimethylformamide (5.0 mL, 50 mM Ni-Aryl concentration), and a stir bar. The red solid immediately dissolved upon addition of the DMF solvent to form a dark red solution. Solutions of the alkyl electrophiles were prepared in individual 4 mL screw-cap vials. Each vial was charged with 0.21 mmol (5 equiv. vs Ni-Aryl) of the electrophile, dimethylformamide (0.55 mL, 370 mM R—X concentration), and a stir bar. BppNiCl₂ (3 mg, 0.001 mmol) was dissolved in a sperate 4 mL screw cap vial with dimethylformamide (0.2 mL, 40 mM bppNiCl₂ solution) to form a light green solution. Finally, the Reduced L2 reaction initiator (92.5 mg, 0.126 mmol) was dissolved in a separate 4 mL vial with dimethylformamide (2.5 mL, 50 mM reduced L2 concentration) to form a dark green/black solution. These solutions were allowed to stir at room temperature for 5 minutes. Aliquots of each stock solution were dispensed in the following order. A 1 mL vial with a magnetic stirbar was first charged with the solution of Ni-Aryl (0.21 mL, 1.0 equiv). The alkyl electrophile was next added via syringe (0.14 mL, 5.0 equiv.). Tertiary alkyl bromides were charged with a solution of bppNiCl₂ (0.050 mL, 0.20 equiv.). Finally, the redox initiator Reduced L2 was charged last (0.21 mL, 1.0 equiv). The vials were sealed and were stirred for 1 hour at room temperature inside the of the nitrogen-filled glove-box. After 1 hour, the organic products were extracted from the crude reaction mixture with ethyl acetate (3 mL) and water (3 mL) and were analyzed by GC.

### Example 3: Scaled Synthesis of (MeBPI) Ni—OAc

A 3 L round-bottom flask flask was charged with^{Me}BPI (100 g, 305 mmol, 1.00 equiv.), Ni(OAc)₂·4H₂O (83.6 g, 336 mmol, 1.10 equiv.), methanol (2.50 L), and a stir bar. The mixture was stirred at room temperature for 24 h then filtered. The golden-yellow solid was collected, suspended in methanol (1.50 L), and stirred for 30 min. This suspension was filtered, and the solid was thoroughly washed with methanol (3×100 mL). Residual solvents were removed under vacuum at 120° C. to yield the title compound as a golden-yellow solid (118 g, 266 mmol, 87%).

### Example 4: Sodium (1Z,3Z)-1,3-bis((4-methylpyridin-2-yl)imino) isoindolin-2-ide (Na^{Me}BPI)

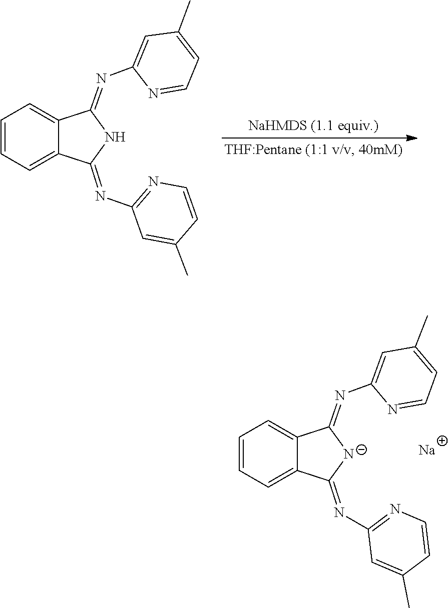 [SMILES: Cc1ccnc(/N=C2\N/C(=N\c3cc(C)ccn3)c3ccccc32)c1 ; Cc1ccnc(/N=C2\[N-]/C(=N\c3cc(C)ccn3)c3ccccc32)c1 ; [Na+]]

In a nitrogen filled glovebox, a 500 mL round-bottomed flask was charged with a magnetic stir bar, ^{Me}BPI (5.00 g, 15.3 mmol, 1.00 equiv.), and a mixture of 1:1 (v/v) pentane:THF (375 mL, 40.0 mM). The solution was allowed to stir for 10 min. NaHMDS (3.08 g, 16.8 mmol, 1.10 equiv.) was added portion wise while stirring. The reaction mixture was allowed to stir for 24 h at room temperature. At this time, an ultrabright yellow suspension had formed, which was filtered and washed with pentane (200 mL) to afford the title compound as a yellow solid (5.04 g, 14.4 mmol, 94%).

### Example 5: Bis((1Z,3Z)—N1,N3-bis(4-methylpyridin-2-yl) isoindoline-1,3-diimine) Nickel (^{Me}BPI)₂Ni

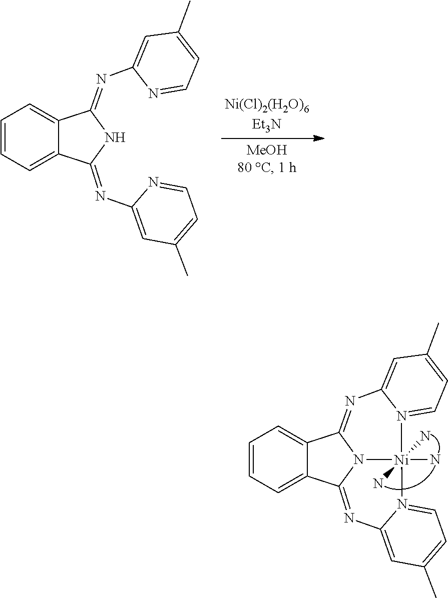

A 250 mL round-bottom flask equipped with a reflux condenser was charged with ^{Me}BPI (6.89 g, 21.0 mmol, 2.00 equiv.), triethylamine (3.67 mL, 26.3 mmol, 2.50 equiv.), NiCl₂(H₂O)₆ (2.50 g, 10.5 mmol, 1.00 eq) and a magnetic stir bar and dissolved in MeOH (150 mL). The reaction was allowed to stir under reflux at 80° C. for 1 h. The solution mixture takes on a brownish/red hue and may completely solidify until the reaction vessel reaches the reflux temperature. Following ~20 min of heating, the solution will completely homogenize. Upon completion and cooling of the reaction, which can be monitored by TLC, solids begin to form near the bottom of the flask. The flask is then placed in the freezer for 1 h allowing for more solids to crash out of solution. The resulting solution is filtered and rinsed with methanol (2×50 mL portions) to produce either a red or purplish/red paste of (^{Me}BPI)₂Ni. Residual solvents are removed under high vacuum to yield the title compound as a red or purplish/red powder (5.25 g, 7.38 mmol, 70% yield). The NMR spectra match those previously reported in the literature.

### Example 6: Na⁺(^{Me}BPI)₂Ni⁻

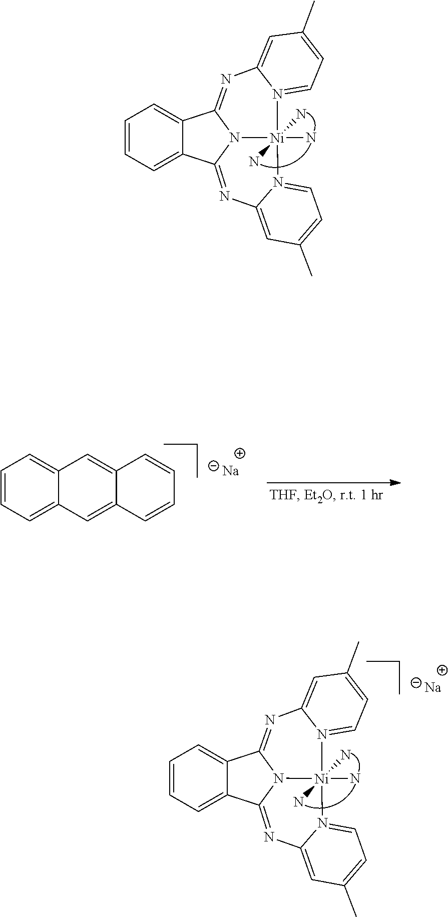

In a nitrogen filled glovebox, a 20 mL vial was charged with Ni(^{Me}BPI) 2 (400. mg, 0.562 mmol), diethyl ether (10 mL), and a magnetic stir bar. To a separate 20 mL vial was added sodium anthracenylide (125 mg, 0.618 mmol, 1.10 equiv.), tetrahydrofuran (10 mL), and a magnetic stir bar. Each mixture was stirred for 5 minutes. The sodium anthracenylide solution was added dropwise to the Ni(^{Me}BPI)₂ while stirring. Upon the full addition of sodium anthracenylide, the reaction was stirred for one additional hour. After this time, a dark green precipitate was observed. The reaction mixture was then filtered, and the green solids were washed with 3×1 mL portions of pentane. The paramagnetic complex was characterized by CV to have a non-zero initial current between −1 and −1.6 V indicative of a reduced species that is oxidized at potentials where only the reduced complex would be oxidized.

### Example 7: General Procedure for the Synthesis Redox Active Esters

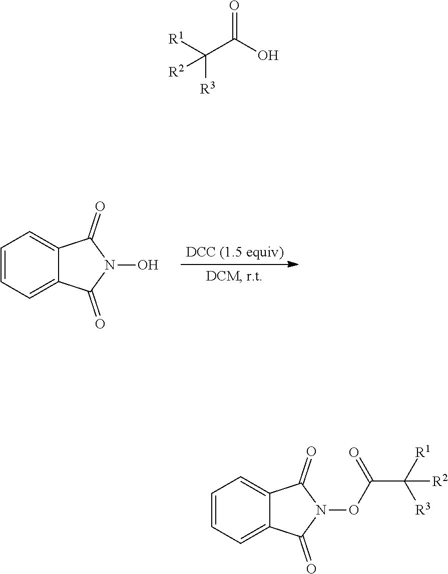

To a 100 mL round-bottomed flask, N-hydroxyphthalimide (1.00 g, 6.13 mmol, 1.00 equiv.), carboxylic acid (1.00 equiv.) was dissolved in anhydrous DCM (300 mM). N,N′-Dicyclohexylcarbodiimide (DCC) (1.90 g, 9.20 mmol, 1.50 equiv.) was added. The reaction was allowed to stir overnight. The reaction mixture was diluted with DCM (50 mL) and washed with saturated NaHCO₃ (3×30 mL), brine (30 mL). The organic layer was allowed to dry with anhydrous Na₂SO₄ and condensed in vacuo to give a white solid. The solid was recrystallized in EtOH to give a pure redox active ester which was then dried under high vacuum and used without further purification. If the redox active ester is an oily paste, the mixture is sonicated while a spatula is used to scratch the wall of the flask to induce solid formation. The flask is then cooled in the freezer for 1 h. The material was used without further purification.

### Example 8: General Procedure for the Synthesis of Alkyl Pyridinium Salts

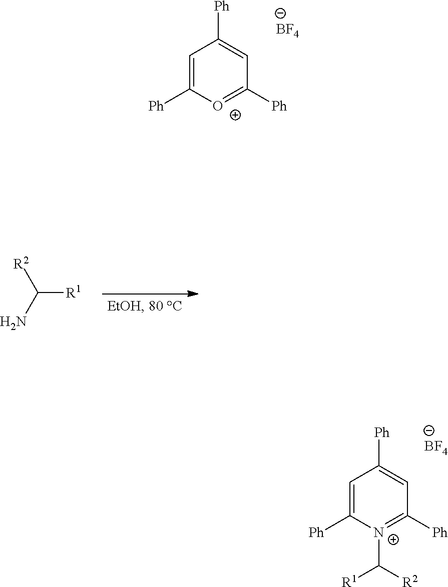

To a 40 mL reaction vial, 2,4,6-triphenylpyrylium tetrafluoroborate (1.98 g, 5.00 mmol, 1.00 equiv.) and the corresponding amine (6.00 mmol, 1.20 equiv.) were dissolved with EtOH (5 mL). The vial was capped and allowed to stir at 80° C. for 6 h. The mixture was then allowed to cool to room temperature. The mixture was diluted with Et₂O (50 mL). If the pyridinium salt failed to precipitate at this point, the solution was placed into a sonicator. While the mixture was sonicated, a spatula was used to scratch the wall of the flask to induce solid formation then placed in the freezer for 1 h. The precipitate was filtered and washed with Et₂O (30 mL). The residue solvent was dried under high vacuum overnight. The product was used for XEC without further purification.

Note: If the pyridinium was not pure or forming the pasty and oily substance during precipitation step, the pyridinium salt was redissolved in DCM (2 mL per 1 g of pyridinium salt). To the solution of diethyl ether (100 mL per 1 g of pyridinium salt) was sonicating, the pyridinium in DCM was added dropwise slowly. The solid was then filtered and washed with diethyl ether.

### Example 9: Development of Electrochemical Oxidative Addition Reactions

In a nitrogen-filled glovebox, a 12 mL screw-cap vial was charged with ^{Me}BPINi—OAc (67.0 mg, 0.15 mmol, 1 equiv.), 4-fluoro-bromobenzene (0.225 mmol, 1.50 equiv.), LiBr (65.0 mg, 0.75 mmol, 5.0 equiv.), DMF (3 mL), and a magnetic stir bar. This yellow slurry was allowed to stir at room temperature for 10 minutes. A Ni foam cathode and a Zn anode were inserted into the test tube. The leads were pierced through a septum cap, and the vial was sealed with a screw-cap. The sealed test tube was removed from the glovebox. The reaction mixture was stirred while a reductive, constant current was applied at the Ni cathode (3 mA, 8.04 mAh, 2.00 equiv. e⁻) at room temperature. The formation of ^{Me}BPI-Ni-Ph-F was monitored by 1° F. NMR spectroscopy.

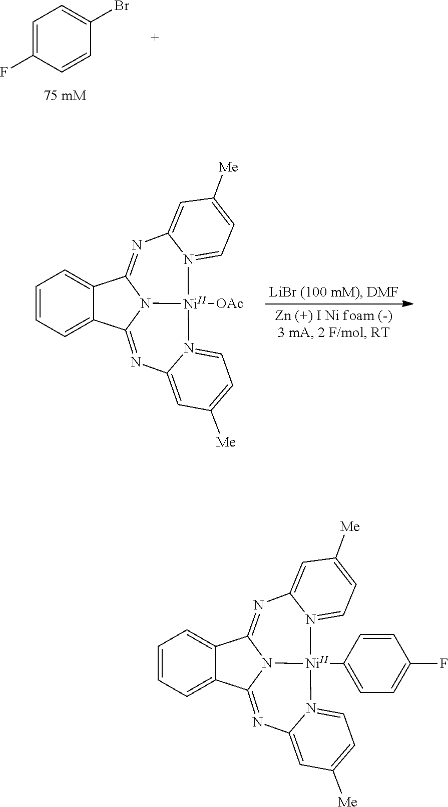

| Entry | Condition | | Yield (%)^{a} |
|---|---|---|---|
| 1 | None | | 70 |
| 2 | No Current with electrode equipped | | 0 |
| 3 | DMA | | 43 |
| 4 | KPF₆ in DMA | | 55 |
| 5 | TBAPF₆ in | Zn (+) \|Ni foam (−) | 62 |
| 6 | DMA | Al (+) \|Ni foam (−) | 50 |
| 7 | | Zn (+) \|RVC (−) | 61 |
| 8 | | Al (+) \|RVC (−) | 58 |

| | | |
|---|---|---|
| 9 | KPF₆ | 57 |
| 10 | TBAPF₆ | 53 |
| 11 | NaBr | 52 |
| 12^{b} | NaI | 62 |
| 13^{b} | LiBr | 87 |
| 14^{b} | LiCl | 75 |
| 15^{b} | LiI | 70 |
| 16^{b} | LiBr, THF:DMF (1:1) | 86 |
| 17^{b} | NaBr, THF:DMF (1:1) | 84 |
| 18^{b} | KBr, THF:DMF (1:1) | 69 |
| 19^{b} | TBABr, THF:DMF (1:1) | 72 |
| 20^{b} | LiCl, THF:DMF (1:1) | 53 |

^{a}Yield monitored by ¹⁹F NMR spectroscopy,
^{b}250 mM electrolyte

### Example 10: General Procedure A: Synthesis of ^{Me}BPINi-Ar from Ni(PPh₃)₄

In a nitrogen-filled glovebox, a 20 mL vial equipped with a magnetic stir bar was charged with Ni(PPh₃)₄ (554 mg, 0.500 mmol, 1.00 equiv.), THF (5 mL, 100 mM). This dark red solution was allowed to stir at room temperature for 5 minutes. Aryl halide (2-3 equiv.) was added and the reaction was allowed to stir for 3 h. Na^{Me}BPI (175 mg, 0.500 mmol, 1.00 equiv.), MeCN (0.5 mL) were added. After addition, the solution was allowed to stir at room temperature for an additional 1 h. The reaction mixture was filtered over basic alumina. The resulting red cake was then washed with THF (5 mL). The filtrate was concentrated in vacuo to afford a dark red solid. The dried solid was dissolved in Et₂O, crashed out with pentane, and filtered to afford the title compound as a bright red solid.

### Example 11: General Procedure B: Synthesis of ^{Me}BPINi-Ar from Ni (Cod) 2

In a nitrogen-filled glovebox, a 20 mL vial equipped with a magnetic stir bar was charged with Ni (cod) 2 (138 mg, 0.500 mmol, 1.00 equiv.), TMEDA (70.0 mg, 0.600 mmol, 1.20 equiv.), aryl halide (0.550 mmol, 1.10 equiv.), toluene (5 mL) (or 1:1 v/v toluene:THF in some case), and a magnetic stir bar. This yellow solid was allowed to stir for 3 h. With stirring, pentane (10 mL) was added slowly, upon which a solid precipitate. The solid was allowed to settle and the liquid was decanted. The remaining solid was dissolved in THF (5 mL). Na^{Me}BPI (175 mg, 0.500 mmol, 1.00 equiv.), MeCN (0.5 mL) were added. After addition, the solution as allowed to stir at room temperature for an additional 1 h. The reaction was filtered over alumina. The resulting red cake was then washed with THF (5 mL). The filtrate was concentrated in vacuo to afford a dark red solid. The dried solid was dissolved in Et₂O, crashed out with pentane, and filtered to afford the title compound as a bright red solid.

### Example 12: General Procedure C: Electrosynthesis of ^{Me}BPINi-Ar from ^{Me}BPINi-OAc

In a nitrogen-filled glove-box, a 12 mL screw-cap vial equipped with a magnetic stir bar was charged with LiBr (108 mg, 1.25 mmol, 5.00 equiv.), ^{Me}BPINiOAc (111 mg, 0.250 mmol, 1.00 equiv.), aryl bromide (0.375 mmol, 1.50 equiv.), and DMF (5 mL). This yellow slurry was allowed to stir at room temperature for 10 min. A Ni foam cathode and a Zn anode were inserted into the test tube, the leads were pierced through a septum cap, and the vial was sealed with a screw-cap. The sealed test tube was removed from the glovebox. The reaction mixture was stirred while a reductive, constant current was applied at the Ni cathode (3 mA, 13.4 mAh, 2.00 equiv. e). After electrolysis, the reaction mixture was transferred to a 40 mL scintillation vial equipped with a stir bar. To this stirring mixture was slowly added degassed water (~30 mL) until a reddish-orange solid precipitated. The resulting slurry was allowed to stir for 10 min at room temperature and then 10 min at 0° C. The precipitated solid was collected by vacuum filtration and washed with water (100 mL) and 0° C. Et₂O (2×5 mL). The resulting orange solid was dissolved in DCM (20 mL) to afford a deep red solution and washed with water (2×30 mL) and brine (30 mL). The red organic layer was dried over Na₂SO₄, filtered, and concentrated by rotary evaporation; the resulting red solid was dissolved in Et₂O. Slow addition of pentane to this stirring mixture affords the product as a dark red or orange solid. Recrystallization was performed for further purification of some complexes.

Alternatively, after electrolysis, the reaction mixture was diluted with benzene (30 mL). The organic layer was washed with degassed water (3×50 mL). The organic layer was dried over Na₂SO₄, filtered, and concentrated by rotary evaporation. The resulting red solid was dissolved in THF (2 mL). Pentane (20 mL) was slowly added to the stirred solution to precipitate the product as a dark red solid.

### Example 13: Coupling Reactions from Isolated ^{Me}BPINi-(Ar) Complexes

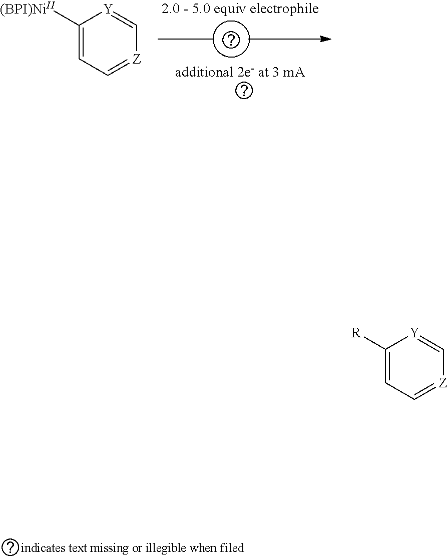

In a nitrogen-filled glovebox, 12 mL screw-cap vial was charged with ^{Me}BPINi-(Ar) (0.12 mmol, 40.0 mM, 1.00 equiv.), electrophile (1.20-5.00 equiv.), 100 mM) or LiBr (65.0 mg, 0.750 mmol, 250. mM) as an electrolyte, DMF (3.0 mL), a magnetic stir bar, a nickel foam cathode, and a zinc anode. The electrodes were threaded through a septum and the reaction was sealed with a screw-cap. The vial was removed from the glovebox and was allowed to stir on a magnetic stir plate at room temperature. A reductive current of 3 mA was applied to the Ni electrode (6.42 mAh, 2.00 equiv. e−). After electrolysis, the product was extracted from the crude reaction mixture with Et₂O (3×25 mL) and water (50 mL). The organic layers were combined and washed with brine (50 mL). The organic layer was dried over Na₂SO₄, filtered, and concentrated by rotary evaporation. The product was purified by flash chromatography on silica gel

### Example 14: Telescoped Coupling Reactions C(Sp²) Electrophiles

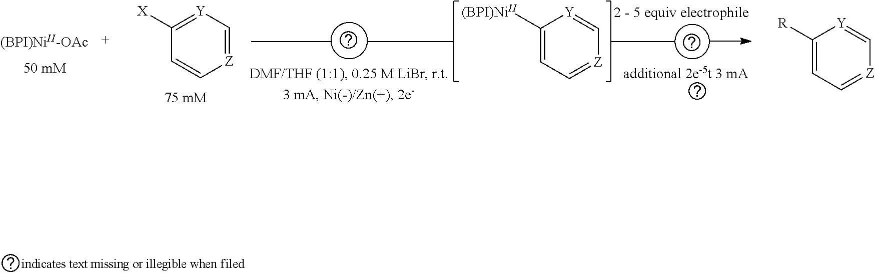

In a nitrogen-filled glove-box, a 12 mL screw-cap vial was charged with ^{Me}BPINi-OAc (67.0 mg, 0.150 mmol, 1.00 equiv.), C(sp²)-electrophile (0.225 mmol, 1.50 equiv.), LiBr (65.0 mg, 0.750 mmol, 5.00 equiv.), DMF (3 mL) (or 1:1 v/v DMF:THF in some instances), and a magnetic stir bar. This yellow slurry was allowed to stir at room temperature for 10 min. A Ni foam cathode and Zn anode were inserted into the test tube, the leads were pierced through a septum cap, and the vial was sealed with a screw-cap. The sealed test tube was removed from the glovebox and the reaction mixture was stirred while a reductive, constant current was applied at the Ni cathode (3 mA, 8.04 mAh, 2.00 equiv. e) at room temperature or 40° C.

To the resulting deep red solution was added the electrophilic coupling partner as a DMF solution (0.300-0.750 mmol, 2-5.0 equiv.). The reaction mixture was stirred while a reductive, constant current was applied at the Ni cathode (3 mA, 8.04 mAh, 2.0 equiv. e″). After electrolysis, the product was extracted from the crude reaction mixture with Et₂O (3×25 mL) and water (50 mL). The organic layers were combined and washed with brine (50 mL). The organic layer was dried over Na₂SO₄, filtered, and concentrated by rotary evaporation. The product was purified by flash chromatography on silica gel.

### Example 16: Scaled Electrosynthesis of ^{Me}BPI-Ni-(4-Methylquinoline) Complex for Telescoped Reactions

In a nitrogen-filled glovebox, a 100 mL media bottle was charged with ^{Me}BPINi-OAc (999 mg, 2.25 mmol, 1.00 equiv.), 2-chloro-4-methylquinoline (600 mg, 3.38 mmol, 1.50 equiv.), LiBr (977 mg, 11.3 mmol, 2.00 equiv.), DMF (45 mL) and a magnetic stir bar. This yellow slurry was allowed to stir at room temperature for 10 min. A Ni foam cathode and Zn anode were inserted through a Teflon liner (see below). The sealed reaction vessel was removed from the glovebox. The reaction mixture was stirred while a reductive, constant current was applied at the Ni cathode (36 mA, 121 mAh, 2.0 equiv. e−) at room temperature.

### Example 17: Ethyl 4-(4-methylquinolin-2-yl) butanoate

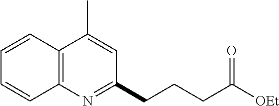

In the nitrogen filled glovebox, a 12 mL reaction was charged with the solution of ^{Me}BPINi-quinoline (3 mL), ethyl 4-bromobutanoate (146 mg, 0.749 mmol, 5.00 equiv.) and subsequently allowed to react under reductive electrolysis (3 mA, 2.0 equiv. e). The title compound was isolated following purification by flash column chromatography (0-25% EtOAc in hexanes) as a yellow oil (40 mg, 0.15 mmol, 104%).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A kit comprising at least two substrate containers,
wherein
the first substrate container includes an organonickel compound of Formula (1): 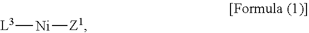
the second substrate container includes an organonickel compound of Formula (2): 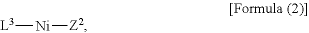
Z¹ has the formula: 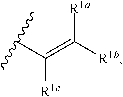
Z² has the formula: 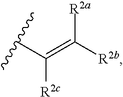
wherein R^{1a}, R^{1b}, R^{1c}, R^{2a}, R^{2b}, and R^{2e} are independently selected from hydrogen, halo, alkyl, heteroalkyl, aryl, or heteroaryl;
wherein Z¹ and Z² are not the same; and
L³Ni has the formula: 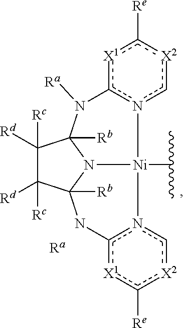
wherein:
c) R^{d} and R^{e} together form a fused benzo ring, and R^{a} and R^{b} together form a double bond; or
d) R^{d} is hydrogen, R^{b} and R^{e} together form a double bond, and R^{a} is C₁₋₄alkyl;
X¹ is N, O, or CR^{f};
X² is null, N, or CR^{g}; and
R^{e} is H, C₁₋₈alkyl, C₁₋₈hydroxylakyl, OC₁₋₈alkyl OC₁₋₈hydroxyalkyl, halo, (CH₂CH₂O)ₙR^{peg} or N(C₁₋₈alkyl)₂; wherein n is 1-20, 2-10, 2-5, 5-10, or 10-20, and R^{Peg} is H or CH₃;
R^{f} is H, C₁₋₈alkyl, OC₁₋₈alkyl, halo, or N(C₁₋₈alkyl)₂; and
R^{g} is H, C₁₋₈alkyl, OC₁₋₈alkyl, halo, or N(C₁₋₈alkyl)₂;
wherein any two or more of R^{e}, R^{f}, and R^{g} may together form a bond.

2. The kit according to claim 1, further comprising at least one electron container including and electron donor.

3. The kit according to claim 2, wherein the electron donor comprises calbatocene, sodium anthracenylide, or a compound of Formula (e): 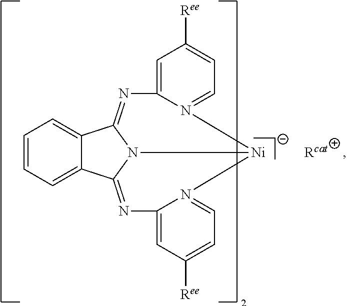
wherein R^{cat} represents an ion in the +1 oxidation state, and R^{ee} is H, C₁₋₄alkyl, OC₁₋₄alkyl, halo, or N(C₁₋₄alkyl)₂.

4. The kit according to claim 2, wherein the kit comprises a single electron container.

5. The kit according to claim 2, wherein the kit comprises the same number of electron containers as substrate containers.

6. The kit according to claim 1, further comprising a compound of Formula (L): 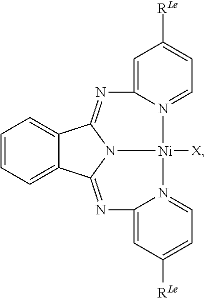
wherein X is halo or OAc; and R^{Le} is H, C₁₋₄alkyl, OC₁₋₄alkyl, halo, N(C₁₋₄alkyl)₂, or (CH₂CH₂O)ₙR^{peg}; wherein n is 1-20, 2-10, 2-5, 5-10, or 10-20, and R^{peg} is H or CH₃;
wherein the compound of Formula (L) is present in the first and second substrate container or is included in a ligand container.

7. The kit according claim 6, wherein the kit comprises a single ligand container.

8. The kit according to claim 6, wherein the kit comprises the same number of ligand containers as substrate containers.

9. The kit according to any preceding claim, wherein the kit comprises a third substrate container that includes an organonickel compound of Formula (3): 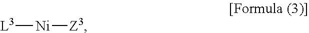
Z³ has the formula: 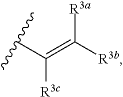
wherein R^{3a}, R^{3b}, and R^{3c} are independently selected from hydrogen, halo, alkyl, aryl, or heteroaryl;
wherein each of Z¹, Z², and Z³ are different.

10. The kit according to any preceding claim, wherein the kit comprises a fourth substrate container that includes an organonickel compound of Formula (4): 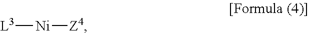
Z⁴ has the formula: 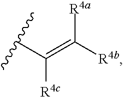
wherein R^{4a}, R^{4b}, and R^{4c} are independently selected from hydrogen, halo, alkyl, heteroalkyl, aryl, or heteroaryl;
wherein each of Z¹, Z², Z³, and Z⁴ are different.

11. The kit according to any preceding claim, wherein the kit comprises a fifth substrate container that includes an organonickel compound of Formula (5): 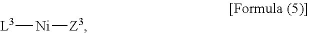
Z⁵ has the formula: 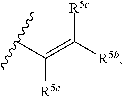
wherein R^{5a}, R^{5b}, and R^{5c} are independently selected from hydrogen, halo, alkyl, heteroalkyl, aryl, or heteroaryl;
wherein each of Z¹, Z², Z³, Z⁴, and Z⁵ are different.

12. The kit according to claim 1, wherein L³-Ni in each case has the formula: 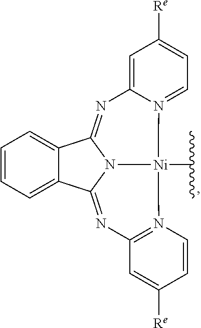
wherein R^{e} is H or C₁₋₄alkyl.

13. The kit according to claim 1, wherein each substrate container includes the organonickel compound in substantially isolated form.

14. The kit according to claim 1, wherein each substrate container includes an organic solvent.

15. The kit according to any preceding claim, wherein each substrate container includes the compound of Formula (L), wherein the compound of Formula (L) will be present in an amount that is no more than 0.1 eq. of the amount of organonickel compound, no more than 0.05 eq. of the amount of organonickel compound, no more than 0.005 eq. of the amount of organonickel compound, or no more than 0.001 eq. of the amount of organonickel compound.
